(19) United States
(12) Patent Application Publication
(10) Pub. No.: US 2021/0405031 A1
(43) Pub. Date: Dec. 30, 2021

(54) SMALL ANIMAL MODELS FOR IN VIVO TESTING OF POLYOMAVIRUS THERAPEUTICS

(71) Applicant: Temple University - of the Commonwealth System of Higher Education, Philadelphia, PA (US)

(72) Inventors: Kamel Khalili, Bala Cynwyd, PA (US); Jennifer Gordon, Philadelphia, PA (US)

(21) Appl. No.: 16/467,813

(22) PCT Filed: Dec. 7, 2017
(86) PCT No.: PCT/US17/65058

§ 371 Date: Jun. 7, 2019

Related U.S. Application Data
(60) Provisional application No. 62/431,473, filed on Dec. 8, 2016.

(51) Int. Cl. G01N 33/50; A01K 67/027

(52) U.S. Cl. CPC G01N 33/5088; A01K 67/0271; A01K 2267/0337; A01K 2227/105; A01K 2207/12

(57) ABSTRACT

Animal models that are permissive for human polyomaviruses and their uses for the screening of candidate agents are described.

### CROSS-REFERENCE TO RELATED APPLICATION

This application claims the benefit of priority to U.S. Provisional Application No. 62/431,473, filed Dec. 8, 2016, the entire contents of each of which are incorporated herein by reference.

### FIELD OF THE INVENTION

Embodiments are directed to animal models that allow for the replication of non-permissive viruses and their use in screening for candidate therapeutic agents.

### BACKGROUND

Polyomaviruses are small non-enveloped double-stranded DNA viruses which display restricted species and cell-type specificity. Up to ten different polyomaviruses have been found in humans that have oncogenic potential and can cause chronic infections. JC virus, or John Cunningham virus (JCV), is a member of the Polyomaviridae family and the causative agent of Progressive Multifocal Leukoencephalopathy (PML), a life-threatening viral infection of the brain. BK virus (BKV) is also a human specific polyomavirus which is responsible for BK nephropathy and loss of graft in renal transplant patients. JCV and BKV are both opportunistic pathogens which infect the human population during early childhood, while the infection is mostly asymptotic. The seroprevalence in adults is about 70-80% (Knowles, ADV. Exp. Med. Biol. 577 (2006), 19-45). The viruses remain latent mostly in the kidney cells of the host until reactivation which occurs in immunosuppressed individuals, such as those suffering from human immunodeficiency virus (HIV) infection, cancer, organ transplantation, hematological malignancies or rarely during autoimmune diseases. Furthermore, immunomodulatory therapies that target immune cells or therapies for conditions such as Multiples Sclerosis (MS) as well as patients with liver or renal impairment, and patients with psoriasis, systemic lupus erythematosus, chronic lymphocytic leukemia (CLL), Hodgkin's lymphoma, and Crohn's disease have an increased risk of incident of PML. JCV infects cerebellar granule cells, oligodendrocytes, astrocytes, and pyramidal cells. So far its primary infection is restricted to kidney, epithelial cells, tonsillar stromal cells, bone marrow, oligodendrocytes, and astrocytes (Frenchy et al., Clin. Microbiol. Rev. 425 (2012), 471-506).

The pathogenesis of PML is characterized by a lytic infection of myelin-forming oligodendrocytes and abortive infection of astrocytes in the absence of a notable immune reaction. However, other central nervous system (CNS) cells such as cerebellar granule neurons can also be infected by JCV. The most frequent symptoms of PML include cognitive impairments, motor dysfunctions, visual deficits, seizures, impaired speech and headaches.

### SUMMARY

Embodiments herein are directed to a model for human polyomavirus replication, e.g. JCV, in rodents using chimeric or transplanted cells which are permissive for human polyomavirus replication. It is an advantage of the invention that the animal models can be easily created in various genetic backgrounds. It is another advantage of the invention that the animal models do not require genetic manipulation to achieve the neuronal phenotypes of various disease states.

Other aspects of the invention are described infra.

### DETAILED DESCRIPTION

Small animal models are needed for pre-clinical testing of therapeutic compounds for the treatment of JC virus induced PML and other polyomavirus-associated diseases. However, human polyomaviruses, including JCV, do not replicate in rodents and thus development of useful rodent models has been limited.

Accordingly, embodiments of the invention are directed to animal models, cells and their uses in identifying candidate agents for the prevention and/or treatment of human polyomavirus infections and associated diseases thereof.

### Definitions

Unless otherwise defined, all terms (including technical and scientific terms) used herein have the same meaning as commonly understood by one of ordinary skill in the art to which this invention belongs. It will be further understood that terms, such as those defined in commonly used dictionaries, should be interpreted as having a meaning that is consistent with their meaning in the context of the relevant art and will not be interpreted in an idealized or overly formal sense unless expressly so defined herein.

As used herein, the singular forms “a”, “an” and “the” are intended to include the plural forms as well, unless the context clearly indicates otherwise. Furthermore, to the extent that the terms “including”, “includes”, “having”, “has”, “with”, or variants thereof are used in either the detailed description and/or the claims, such terms are intended to be inclusive in a manner similar to the term “comprising.”

As used in this specification and the appended claims, the term “or” is generally employed in its sense including “and/or” unless the content clearly dictates otherwise.

The term “about” or “approximately” means within an acceptable error range for the particular value as determined by one of ordinary skill in the art, which will depend in part on how the value is measured or determined, i.e., the limitations of the measurement system. For example, “about” can mean within 1 or more than 1 standard deviation, per the practice in the art. Alternatively, “about” can mean a range of up to 20%, up to 10%, up to 5%, or up to 1% of a given value or range. Alternatively, particularly with respect to biological systems or processes, the term can mean within an order of magnitude within 5-fold, and also within 2-fold, of a value. Where particular values are described in the application and claims, unless otherwise stated the term “about” meaning within an acceptable error range for the particular value should be assumed.

The term “animal” is used herein to include all vertebrate animals, except humans. It also includes an individual animal in all stages of development, including embryonic and fetal stages. The term “animal model” as used herein refers to any non-human animals directly or indirectly manipulated, e.g. transplanted cells or tissues, or grafted with cells or tissue). In one embodiment, the animal model is an immuno-compromised non-human animal capable of receiving and supporting transplanted cells, tissues, or a xenograft without mounting a graft-rejection immune response. An “immunocompromised” animal can either be an immunodeficient animal which is genetically deprived of endogenous T cells, B cells, NK cells or a combination thereof. Alternatively, an animal can be immunosuppressed by biological or chemical means. Such biological or chemical means include, without limitation, immuno-suppression by repeated treatment with irradiation, mitomycin C, cyclosporine, anti-Asialo GM1 antibody, or other immuno-suppressive agents or treatments well known in the art. In some of the embodiments, the animals or animal models of present disclosure are immunodeficient. The term “immunodeficient” is used herein to describe the animal whose endogenous immune system has been partly or completely compromised, such that it does not generate sufficient immune response to reject a foreign graft (such as a foreign cell or a tissue) and therefore is capable of accepting and supporting the foreign graft as self. In certain embodiments, the immunodeficient animal is depleted of active endogenous T cells, active endogenous B cells and active endogenous Natural Killer cells. Examples of immuno-deficient animals include, for example: T lymphocytes deficient animals (e.g. BALB/c nude mice, C57BL nude mice, NIH nude mice, nude rat, etc.); B lymphocytes deficient animals (e.g. CBA/N mice); NK cell deficient animal (e.g. Beige mice); combined immunodeficient animal (e.g. severe combined immune-deficient (SCID) mice (combined T and B lymphocytes deficient), Beige/Nude (combined T lymphocytes and NK cells deficient), SCID (Severe Combined Immune Deficiency, also known as Prkdc^{scid}) Beige/NOD (Non-Obese Diabetes) SCID mice (combined T, B lymphocytes and NK cells deficient)), and animals which are treated or manipulated to have an immune system which resembles that in any of the above-mentioned immuno-deficient animals. In certain embodiments, the immunodeficient animals are NOD SCID mice further depleted of Interleukin 2 receptor gamma chain i.e., NSG (NOD-SCID-Gamma) mice, (Shultz L D; Lyons B L; Burzenski L M et al., 2005, J. Immunol. 174 (10): 6477-89; Shultz L D; Schweitzer P A; Christianson S W et al., 1995, J. Immunol. 154 (1): 180-91). NSG mice are deficient in multiple cytokine signaling pathways and hence deficient in innate immunity, which permit the engraftment of a wide range of primary human cells, and enable sophisticated modeling of many areas of human biology and disease in such type of animal model. Examples of different strains of NSG mice include, for example: original NSG mice (developed by The Jackson Laboratory), NPG mice (NOD-Prkdc^{scid} Il2rg^{null} mice developed by Beijing Vitalstar Biotechnology), NOG mice (NOD/Shi-scid/IL-2Rγ^{null} mice developed by Central Institute for Experimental Animals (CIEA)), NCG (NOD-Prkdc^{em26cas9d52}Il2rg^{em26cas9d22}Nju mice developed by Model Animal Research Center of Nanjing University). Different strains of NSG mice are by so far most highly immunodeficient mice available, they have longer life span than NOD SCID mice which enables them for long term observations, they have basically no rejection to human derived cells or tissue and are with extremely low amount of active NK cells.

A “candidate agent” as used herein refers to any agent that is a candidate to treat a disease or symptom thereof. The term “agent” is meant to encompass any molecule, chemical entity, composition, drug, therapeutic agent, chemotherapeutic agent, or biological agent capable of preventing, ameliorating, or treating a disease or other medical condition. The term includes small molecule compounds, antisense reagents, siRNA reagents, antibodies, enzymes, peptides organic or inorganic molecules, natural or synthetic compounds and the like. An agent can be assayed in accordance with the methods of the invention at any stage during clinical trials, during pre-trial testing, or following FDA-approval.

As used herein, the terms “comprising,” “comprise” or “comprised,” and variations thereof, in reference to defined or described elements of an item, composition, apparatus, method, process, system, etc. are meant to be inclusive or open ended, permitting additional elements, thereby indicating that the defined or described item, composition, apparatus, method, process, system, etc. includes those specified elements—or, as appropriate, equivalents thereof—and that other elements can be included and still fall within the scope/definition of the defined item, composition, apparatus, method, process, system, etc.

The terms “determining”, “measuring”, “evaluating”, “detecting”, “assessing” and “assaying” are used interchangeably herein to refer to any form of measurement, and include determining if an element is present or not. These terms include both quantitative and/or qualitative determinations. Assessing may be relative or absolute. “Assessing the presence of” includes determining the amount of something present, as well as determining whether it is present or absent.

The term “eradication” of a virus, e.g. human polyomavirus, as used herein, means that that virus is unable to replicate, the genome is deleted, fragmented, degraded, genetically inactivated, or any other physical, biological, chemical or structural manifestation, that prevents the virus from being transmissible or infecting any other cell or subject resulting in the clearance of the virus in vivo. In some cases, fragments of the viral genome may be detectable, however, the virus is incapable of replication, or infection etc.

An “effective amount” as used herein, means an amount which provides a therapeutic or prophylactic benefit.

The term “small molecule” refers to a molecule of a size comparable to those organic molecules generally used in pharmaceuticals. The term excludes biological macromolecules (e.g., proteins, nucleic acids, etc.). Preferred small organic molecules range in size up to about 5000 Da, up to about 2000 Da, and up to about 1000 Da.

To “treat” a disease as the term is used herein, means to reduce the frequency or severity of at least one sign or symptom of a disease or disorder experienced by a subject. Treatment of a disease or disorders includes the eradication of a virus.

“Treatment” is an intervention performed with the intention of preventing the development or altering the pathology or symptoms of a disorder. Accordingly, “treatment” refers to both therapeutic treatment and prophylactic or preventative measures. “Treatment” may also be specified as palliative care. Those in need of treatment include those already with the disorder as well as those in which the disorder is to be prevented. Accordingly, “treating” or “treatment” of a state, disorder or condition includes: (1) eradicating the virus; (2) preventing or delaying the appearance of clinical symptoms of the state, disorder or condition developing in a human or other mammal that may be afflicted with or predisposed to the state, disorder or condition but does not yet experience or display clinical or subclinical symptoms of the state, disorder or condition; (3) inhibiting the state, disorder or condition, i.e., arresting, reducing or delaying the development of the disease or a relapse thereof (in case of maintenance treatment) or at least one clinical or subclinical symptom thereof; or (4) relieving the disease, i.e., causing regression of the state, disorder or condition or at least one of its clinical or subclinical symptoms. The benefit to an individual to be treated is either statistically significant or at least perceptible to the patient or to the physician.

The term “xenograft” as used herein refers to tissue or cells taken from or derived from a donor which is a species different from the animal model, and are suitable for being grafted into the animal model. In some embodiments, the donor of the xenograft is human. In some embodiments, the human xenograft is derived from a human patient having the disease.

Animal Models

Embodiments of the invention are directed to animal models that are permissive for human polyomavirus and the associated diseases of human polyomavirus infections thereof. In some embodiments, the animal model comprises an immunodeficient animal. In some embodiments, the animal is a mammal. In some embodiments, the mammal is a rodent, such as a mouse, a rat, a guinea pig, or a hamster. In some embodiments, the animal is mouse, a rat, a guinea pig, a hamster, a dog, a pig, or a primate.

In some embodiments, the animal is a mouse. Mouse models with a humanized immune system such as immunocompromised and sub-lethally irradiated mice reconstituted with CD34⁺ human fetal liver cells (huNSG mice) or reconstituted with human thymus and human lymphocytes (BLT mice) produce functional circulating CD19⁺ human B cells. BLT mice inoculated with JCV remain asymptomatic following inoculation but JCV DNA was detected in both blood and urine and mice generated both humoral and cellular immune responses against JCV concomitant with expression of the immune exhaustion marker, PD-1, on lymphocytes consistent with a response to an infection. These mice can be infected with JCV and used as a model for in vivo replication of the virus in B cells.

In some embodiments, the animal model is an immunodeficient rodent comprising: (a) transplanted cells permissive for human polyomavirus replication; and/or (b) a human xenograft comprising a human cell or tissue. In other embodiments, the animal model is a rodent comprising (a) transplanted cells permissive for human polyomavirus replication; and/or (b) a human xenograft comprising a human cell or tissue. In other embodiments, the animal model is immunodeficient comprising (a) transplanted cells permissive for human polyomavirus replication; and/or (b) a human xenograft comprising a human cell or tissue. In other embodiments, the animal model comprises (a) transplanted cells permissive for human polyomavirus replication; and/or (b) a human xenograft comprising a human cell or tissue.

In other embodiments, the animal model is a knock-in or knock-out animal. A knock-in animal can have genetic material introduced which renders the animal model permissive for polyomavirus replication and infection. A knock-out may have genetic material deleted which allows for the polyomavirus to replicate and infect cells. In certain embodiments, the animal model has genetic material introduced and certain genetic material removed to produce an animal which allows the polyomavirus to replicate and infect. Knock-in and knock-out animals can be produced by any methods known in the art. Selection of genetic material can include promoters, enhancers, removal of suppressors, etc., which would allow the virus to replicate and infect cells.

In some embodiments, cells permissive for human polyomavirus replication comprise: human cells, humanized cells, primate cells, transformed cells, cell-lines, tumor cells, stem cells, hybrid cells, cells engineered to spontaneously shed a polyomavirus, or combinations thereof. In some embodiments, the cells comprise sequences from a human polyomavirus and express the viral early transcript under the control of the human polyomavirus promoter. For example, JCV strains were inoculated into newborn Syrian golden hamsters, newborn Sprague-Dawley rats, and owl and squirrel monkeys by various routes including intracerebral, intraocular, and intraperitoneal. Cell lines derived from tumor tissue harvested from these animal models are available in the laboratory. These cell lines contain sequences of the virus and express the viral early transcript under the control of the JCV promoter and several of these cell lines support replication of JC virus.

In one embodiment, cells of primate or human origin neural which support JCV replication are transplanted into the flank or the brain of immunocompromised mice, e.g. Owl 586 cells which spontaneously shed JC virus inoculated into Nude mice. Transplanted cells support in vivo replication of JCV at the site of inoculation.

In another embodiment, renal cells of primate or human origin cells which support JCV replication are transplanted into the flank or kidney subcapsular region of immunocompromised mice, e.g. COS-7 cells which are infected with JC virus inoculated into nude mice. Transplanted cells support in vivo replication of JCV at the site of inoculation.

In another embodiment, intravenous inoculation of huNSG or BLT mice containing functional circulating CD19⁺ human B cells with JC virus. Engrafted human B cells support replication of JCV in circulating blood and lymphoid tissues.

In some embodiments, the cells permissive for human polyomavirus replication or the human xenograft are transplanted or grafted to the animal intravenously, or subcutaneously, or intramuscularly, or intraperitoneally.

The following examples of cells permissive for human polyomavirus are meant to be illustrative and are not to be construed as a limitation of the invention in any way:

Owl monkey cells. New World primates, owl and squirrel monkeys, inoculated with purified JCV developed glial neoplasias, including glioblastoma multiforme and astrocytoma. With one exception, no evidence of viral replication was observed, although the expression of the early gene, T-antigen, was detected by immunohistochemistry or by Western blot analysis. Cells cultured from an astrocytoma arising in one owl monkey inoculated with JCV were found to produce spontaneously infectious JC viral particles, although some rearrangement of the viral regulatory region may have occurred (Major et al., 1987). These cells can be propagated in immunocompromised mice to allow spontaneous JCV replication in vivo.

SV40-transformed monkey cells. SV40-transformed monkey cells support JCV replication and can be transplanted into immunocompromised mice support JCV replication. The SV40-transformed monkey glial cell lines, SVG and its derivative, SVG-A, as well as SV40-transformed monkey kidney CV-1 cells, called COS-7, can be used to propagate JC virus strains in culture. The PML derived strains are typically cultured in SVG or SVG-A cells while the archetype (kidney derived strain) is propagated in COS-7 monkey kidney cells. The SV40 T-antigen boosts replication of JC virus in trans. SVG-A and COS-7 cells can be propagated in immunocompromised mice to allow JCV replication in vivo.

JCV-transformed hamster cells and mouse cells. JCV-transformed hamster and mouse cells express JCV early transcripts and can be transplanted into immunocompromised mice. Hamsters inoculated with JCV developed neuronal and glial-origin tumors, most frequently medulloblastoma, peripheral neuroblastoma, astrocytoma, and primitive neuroectodermal tumors. Similar studies performed using newborn Sprague-Dawley rats resulted in the induction of primitive neuroectodermal tumors. Studies using transgenic mice expressing the JCV early transcript under the control of the JCV promoter also induced a broad range of tumors. Tumor tissues showed the expression of the viral T-antigen in the nucleus of the tumor cells. However, no signs of viral replication including the expression of the viral late capsid proteins by immunohistochemistry or virion formation by electron microscopy were observed. HJC cells and their derivatives (HJC series) were cultured from tumors harvested from hamsters inoculated with JCV (see Raj et al., 1995 for details). BSB7 cells and parallel clones (BSB7 series) were derived from tumors harvested from transgenic mouse models (see Krynska et al., 2000 for details). Both sets of these transformed cell lines stably express the viral early transcript and are tumorigenic when inoculated into immunocompromised (Nude) mice. (While the hamster and mouse cells do not support viral replication, they stably express the early mRNA and can be used to assess therapeutics aimed at blocking the early stages of the JCV replication cycle).

Human glial progenitor cells. These cells can be propagated in immunocompromised mice to allow spontaneous JCV replication in vivo. Studies by Kondo et al., showed a mouse model was generated by engrafting bipotential human glial progenitor cells (GPCs) prepared from human fetal brain tissue into neonatal immunodeficient and myelin-deficient mice (Rag2^{-/-} Mbp^{shi/shi}). The forebrain glial populations of these mice became substantially humanized with age. When injected intracerebrally with JCV, productive infection occurred and subsequent demyelination.

The animal models of the invention are especially useful as pharmacodynamic models to test the therapeutic efficacy of agents targeting human polyomaviruses and diseases associated with a human polyomavirus infection, e.g. PML.

The animal models of the invention can also be used as research tools for the discovery and development of therapeutic products for preventing and/or treating a virus infection, e.g. human polyomavirus, and associated diseases thereof, e.g. PML. The models may be useful in various aspects of drug discovery and investigation, including without limitation the initial identification of an agent as a drug candidate, the confirmation of activity of a drug candidate, and the identification of activity in an existing pharmaceutical product.

Candidate Agents

Candidate agents may be a protein, polypeptide, peptide, oligonucleotide, polynucleotide, lipid, organic or inorganic molecule, carbohydrate, or other compound which may inhibit the human polyomavirus replication and/or eradication of the human polyomavirus from the infected cell or tissue. Such candidate agents include agents which are natural products or which are prepared synthetically. Non-limiting examples include endonucleases, polypeptides, peptidomimetics, pharmacophores, small molecules, the compounds found in the U.S. Pharmacopoeia, and the products of combinatorial chemical synthesis. Candidate pharmaceuticals include molecules for which no function is known, but which have structural similarity to known compounds with one or more known functions.

Candidate agents include numerous chemical classes, though typically they are organic compounds including small organic compounds, nucleic acids including oligonucleotides, and peptides. Small organic compounds suitably may have e.g. a molecular weight of more than about 40 or 50 yet less than about 2,500. Candidate agents may comprise functional chemical groups that interact with proteins and/or DNA.

Candidate agents may be obtained from a wide variety of sources including libraries of synthetic or natural compounds. For example, numerous means are available for random and directed synthesis of a wide variety of organic compounds and biomolecules, including expression of randomized oligonucleotides. Alternatively, libraries of natural compounds in the form of e.g. bacterial, fungal and animal extracts are available or readily produced.

Endonucleases: Screening of endonucleases for the inactivation, deletion and/or eradication of the human polyomavirus, such as JCV, is also contemplated. Any suitable nuclease system can be used including, for example, Argonaute family of endonucleases, clustered regularly interspaced short palindromic repeat (CRISPR) nucleases, zinc-finger nucleases (ZFNs), transcription activator-like effector nucleases (TALENs), meganucleases, other endo- or exo-nucleases, or combinations thereof. See Schiffer, 2012, J Virol 88(17):8920-8936, incorporated by reference.

One preferred gene editing means for eliminating, for example, latent JCV is RNA-guided CRISPR technology. In a CRISPR system, CRISPR clusters encode spacers, which are sequences complementary to target sequences (“protospacers”) in a viral nucleic acid, or in another nucleic acid to be targeted. CRISPR clusters are transcribed and processed into mature CRISPR RNAs (crRNAs). CRISPR clusters also encode CRISPR associated (Cas) proteins, which include DNA endonucleases. The crRNA binds to target DNA sequence, whereupon the Cas endonuclease cleaves the target DNA at or adjacent to the target sequence.

One useful CRISPR system includes the CRISPR associated endonuclease Cas9. Cas9 is guided by a mature crRNA that contains about 20-30 base pairs (bp) of spacer and a trans-activated small RNA (tracrRNA) that serves as a guide for ribonuclease III-aided processing of pre-crRNA. The crRNA:tracrRNA duplex directs Cas9 to target DNA via complementary base pairing between the spacer on the crRNA and the target sequence on the target DNA. Cas9 recognizes a trinucleotide (NGG) protospacer adjacent motif (PAM) to decide the cut site (the 3rd nucleotide from PAM). The crRNA and tracrRNA can be expressed separately or engineered into an artificial chimeric small guide RNA (sgRNA) via a synthetic stem loop (AGAAAU) to mimic the natural crRNA/tracrRNA duplex. Such sgRNAs, can be synthesized or in vitro transcribed for direct RNA transfection, or they can be expressed in situ, e.g. from U6 or H1-promoted RNA expression vectors. The term “guide RNA” (gRNA) will be used to denote either a crRNA:tracrRNA duplex or an sgRNA. It will be understood that the term “gRNA complementary to” a target sequence indicates a gRNA whose spacer sequence is complementary to the target sequence.

Chemical Libraries: Developments in combinatorial chemistry allow the rapid and economical synthesis of hundreds to thousands of discrete compounds. These compounds are typically arrayed in moderate-sized libraries of small molecules designed for efficient screening. Combinatorial methods, can be used to generate unbiased libraries suitable for the identification of novel compounds. In addition, smaller, less diverse libraries can be generated that are descended from a single parent compound with a previously determined biological activity. In either case, the lack of efficient screening systems to specifically target therapeutically relevant biological molecules produced by combinational chemistry such as inhibitors of important enzymes hampers the optimal use of these resources.

A combinatorial chemical library is a collection of diverse chemical compounds generated by either chemical synthesis or biological synthesis, by combining a number of chemical “building blocks,” such as reagents. For example, a linear combinatorial chemical library, such as a polypeptide library, is formed by combining a set of chemical building blocks (amino acids) in a large number of combinations, and potentially in every possible way, for a given compound length (i.e., the number of amino acids in a polypeptide compound). Millions of chemical compounds can be synthesized through such combinatorial mixing of chemical building blocks.

A “library” may comprise from 2 to 50,000,000 diverse member compounds. Preferably, a library comprises at least 48 diverse compounds, preferably 96 or more diverse compounds, more preferably 384 or more diverse compounds, more preferably, 10,000 or more diverse compounds, preferably more than 100,000 diverse members and most preferably more than 1,000,000 diverse member compounds. By “diverse” it is meant that greater than 50% of the compounds in a library have chemical structures that are not identical to any other member of the library. Preferably, greater than 75% of the compounds in a library have chemical structures that are not identical to any other member of the collection, more preferably greater than 90% and most preferably greater than about 99%.

The preparation of combinatorial chemical libraries is well known to those of skill in the art. For reviews, see Thompson et al., Synthesis and application of small molecule libraries, Chem Rev 96:555-600, 1996; Kenan et al., Exploring molecular diversity with combinatorial shape libraries, Trends Biochem Sci 19:57-64, 1994; Janda, Tagged versus untagged libraries: methods for the generation and screening of combinatorial chemical libraries, Proc Natl Acad Sci USA. 91:10779-85, 1994; Lebl et al., One-bead-one-structure combinatorial libraries, Biopolymers 37:177-98, 1995; Eichler et al., Peptide, peptidomimetic, and organic synthetic combinatorial libraries, Med Res Rev. 15:481-96, 1995; Chabala, Solid-phase combinatorial chemistry and novel tagging methods for identifying leads, Curr Opin Biotechnol. 6:632-9, 1995; Dolle, Discovery of enzyme inhibitors through combinatorial chemistry, Mol Divers. 2:223-36, 1997; Fauchere et al., Peptide and nonpeptide lead discovery using robotically synthesized soluble libraries, Can J. Physiol Pharmacol. 75:683-9, 1997; Eichler et al., Generation and utilization of synthetic combinatorial libraries, Mol Med Today 1: 174-80, 1995; and Kay et al., Identification of enzyme inhibitors from phage-displayed combinatorial peptide libraries, Comb Chem High Throughput Screen 4:535-43, 2001.

Other chemistries for generating chemical diversity libraries can also be used. Such chemistries include, but are not limited to, peptoids (PCT Publication No. WO 91/19735); encoded peptides (PCT Publication WO 93/20242); random bio-oligomers (PCT Publication No. WO 92/00091); benzodiazepines (U.S. Pat. No. 5,288,514); diversomers, such as hydantoins, benzodiazepines and dipeptides (Hobbs, et al., Proc. Nat. Acad. Sci. USA, 90:6909-6913 (1993)); vinylogous polypeptides (Hagihara, et al., J. Amer. Chem. Soc. 114:6568 (1992)); nonpeptidal peptidomimetics with β-D-glucose scaffolding (Hirschmann, et al., J. Amer. Chem. Soc., 114:9217-9218 (1992)); analogous organic syntheses of small compound libraries (Chen, et al., J. Amer. Chem. Soc., 116:2661 (1994)); oligocarbamates (Cho, et al., Science, 261:1303 (1993)); and/or peptidyl phosphonates (Campbell, et al., J. Org. Chem. 59:658 (1994)); nucleic acid libraries (see, Ausubel, Berger and Sambrook, all supra); peptide nucleic acid libraries (see, e.g., U.S. Pat. No. 5,539,083); antibody libraries (see, e.g., Vaughn, et al., Nature Biotechnology, 14(3):309-314 (1996) and PCT/US96/10287); carbohydrate libraries (see, e.g., Liang, et al., Science, 274:1520-1522 (1996) and U.S. Pat. No. 5,593,853); small organic molecule libraries (see, e.g., benzodiazepines, Baum C&E News, January 18, page 33 (1993); isoprenoids (U.S. Pat. No. 5,569,588); thiazolidinones and metathiazanones (U.S. Pat. No. 5,549,974); pyrrolidines (U.S. Pat. Nos. 5,525,735 and 5,519,134); morpholino compounds (U.S. Pat. No. 5,506,337); benzodiazepines (U.S. Pat. No. 5,288,514); and the like.

Devices for the preparation of combinatorial libraries are commercially available (see, e.g., 357 MPS, 390 MPS, Advanced Chem. Tech, Louisville Ky., Symphony, Rainin, Woburn, Mass., 433A Applied Biosystems, Foster City, Calif., 9050 Plus, Millipore, Bedford, Mass.). In addition, numerous combinatorial libraries are themselves commercially available (see, e.g., ComGenex, Princeton, N.J., Asinex, Moscow, Ru, Tripos, Inc., St. Louis, Mo., ChemStar, Ltd., Moscow, RU, 3D Pharmaceuticals, Exton, Pa., Martek Bio sciences, Columbia, Md., etc.).

Assessment of Candidate Agents: In certain embodiments, the present disclosure provides methods of assessing effect of a candidate agent on the viability and/or eradication of a human polyomavirus and/or associated disease thereof. In some embodiments, the methods comprising: a) obtaining an animal model embodied herein; b) administering the candidate agent to the animal model; and c) determining the effect of the candidate agent on the animal model.

In some embodiments, a method of identifying a candidate agent for inhibiting a human polyomavirus infection, or replication in vivo, comprising administering to an animal embodied herein, a candidate agent and assaying for human polyomavirus infection or replication.

The method of assessing the effect of a candidate agent comprises any type of assays, including, for example, cell based assays, immunoassays, immunoblotting assays, gel assays, PCR, hybridization assays, measuring plaque forming units, and the like.

The candidate agent is administered to supply a desired therapeutic dose to promote a desired therapeutic response to the therapeutic area. By “desired therapeutic response” is intended an improvement in the condition or in the symptoms associated with the condition, including the inhibition of virus replication, deletion of virus genetic material, etc. In preferred aspects, the animals treated with a composition comprising a candidate agent are compared to a control group of animals not treated with a candidate agent. Such a control group may be animals matched in physiological characteristics (e.g., age, strain, genetic background, etc.) that has not received the composition that comprises a candidate agent. In certain aspects, the control group not treated with the candidate agent receives no composition. In other aspects, the control group not treated with a candidate agent receives a composition with all or a subset of the elements used in the composition comprising the candidate agent except for the candidate agent itself. These control groups allow the identification of a physiologically significant effect of the control agent by comparison to matched animals that do not receive the control agent.

The candidate agents can be formulated in a unit dosage such as a solution, suspension or emulsion, in association with a pharmaceutically acceptable carrier. Such carriers are inherently nontoxic and nontherapeutic. Examples of such carriers are saline, Ringer's solution, dextrose solution, and Hanks' solution. Nonaqueous carriers such as fixed oils and ethyl oleate may also be used. The vehicle may contain minor amounts of additives such as substances that enhance chemical stability, including buffers and preservatives.

Various methods of delivery can be used to deliver the candidate agent to the region of interest, and will in part be dependent upon the agent and its bioavailability. For example, small molecules or other agents that are bioavailable may be administered orally, whereas protein-based agents are generally but not exclusively administered parenterally. Certain agents may be administered systemically, while others may be more beneficial with a local delivery. The method of delivery will be apparent to one skilled in the art upon reading the specification, and can be determined in view of the specific properties of the candidate agent.

A pharmaceutically effective amount of a candidate agent of the invention is administered to a subject. By “pharmaceutically effective amount” is intended an amount that is useful in the treatment of a disease or condition. In this manner, a pharmaceutically effective amount of the candidate agent can be introduced to the region of interest in a non-human animal model of the invention. By “therapeutically effective dose or amount” or “effective amount” is meant an amount of the candidate agent that, when administered, brings about a positive therapeutic response with respect to human polyomavirus infection and/or associated diseases thereof, e.g. PML. In some embodiments of the invention, the therapeutically effective dose is in the range from about 0.1 μg/kg to about 100 mg/kg body weight, about 0.001 mg/kg to about 50 mg/kg, about 0.01 mg/kg to about 30 mg/kg, about 0.1 mg/kg to about 25 mg/kg, about 1 mg/kg to about 20 mg/kg, about 3 mg/kg to about 15 mg/kg, about 5 mg/kg to about 12 mg/kg, about 7 mg/kg to about 10 mg/kg or any range of value therein. It is recognized that the method of treatment may comprise a single administration of a therapeutically effective dose or multiple administrations of a therapeutically effective dose.

It is understood that the effective amount may vary depending on the nature of the effect desired, frequency of treatment, any concurrent treatment, the health, weight of the recipient, and the like. See, e.g., Berkow et al., eds., Merck Manual, 16th edition, Merck and Co., Rahway, N.J. (1992); Goodman et al., eds., Goodman and Oilman's The Pharmacological Basis of Therapeutics, 8th edition, Pergamon Press, Inc., Elmsford, N.Y. (1990); Avery's Drug Treatment: Principles and Practice of Clinical Pharmacology and Therapeutics, 3rd edition, ADIS Press, LTD., Williams and Wilkins, Baltimore, Md. (1987), Ebadi, Pharmacology, Little, Brown and Co., Boston (1985), Katzung, Basic and Clinical Pharmacology, Appleton and Lange, Norwalk, Conn. (1992), which references and references cited therein, are entirely incorporated herein by reference.

The candidate agent may be contained in a pharmaceutically-acceptable carrier, and supplementary active compounds can also be incorporated into the candidate agents. A composition comprising a candidate agent is formulated to be compatible with its intended route of administration. Examples of routes of administration include intravenous, intraarterial, intracoronary, parenteral, subcutaneous, subdermal, subcutaneous, intraperitoneal, intraventricular infusion, infusion catheter, balloon catheter, bolus injection, direct application to tissue surfaces during surgery, or other convenient routes. The composition can also be injected into an ischemic area of interest, to pharmacologically start the process of blood vessel growth and collateral artery formation.

Solutions or suspensions used for such administration can include other components such as sterile diluents like water for dilution, saline solutions, polyethylene glycols, glycerin, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates, and agents for the adjustment of tonicity such as sodium chloride or dextrose. The pH can be adjusted with acids or bases, such as hydrochloric acid or sodium hydroxide. The composition can be enclosed in ampoules, disposable syringes or multiple dose vials made of glass or plastic.

Compositions comprising candidate agents suitable for injectable use include sterile aqueous solutions (where water soluble) or dispersions and sterile powders for the extemporaneous preparation of sterile injectable solutions or dispersion. For intravenous administration, suitable carriers include physiological saline, bacteriostatic water, or phosphate buffered saline (PBS). In all cases, the composition must be sterile and should be fluid to the extent possible. It must be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants.

Prevention of the action of microorganisms in the compositions can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as mannitol, sorbitol, sodium chloride in the composition. Prolonged absorption of the injectable compositions can be brought about by including in the composition an agent which delays absorption, for example, aluminum monostearate and gelatin. Sterile injectable solutions can be prepared by incorporating an agent in the required amount in an appropriate solvent with a selected combination of ingredients, followed by filter sterilization. Generally, dispersions are prepared by incorporating an agent into a sterile vehicle that contains a basic dispersion medium and the required other ingredients from those enumerated above. In the case of sterile powders for the preparation of sterile injectable solutions, methods of preparation are vacuum drying and freeze-drying that yields a powder of the active ingredient plus any additional desired ingredient from a previously sterile-filtered solution thereof.

While various embodiments of the present invention have been described above, it should be understood that they have been presented by way of example only, and not limitation. Numerous changes to the disclosed embodiments can be made in accordance with the disclosure herein without departing from the spirit or scope of the invention. Thus, the breadth and scope of the present invention should not be limited by any of the above described embodiments.

All documents mentioned herein are incorporated herein by reference. All publications and patent documents cited in this application are incorporated by reference for all purposes to the same extent as if each individual publication or patent document were so individually denoted. By their citation of various references in this document, Applicants do not admit any particular reference is “prior art” to their invention. Embodiments of inventive compositions and methods are illustrated in the following examples.

### EXAMPLES

### Example 1: PML Animal Models

Small animal models are provided for pre-clinical testing of therapeutic compounds for the treatment of JC virus induced PML and other polyomavirus-associated diseases.

1) In one example of an animal model, cells of primate or human origin neural cells which support JCV replication are transplanted into the flank or the brain of immunocompromised mice, e.g. Owl 586 cells which spontaneously shed JC virus inoculated into Nude mice. Transplanted cells support in vivo replication of JCV at the site of inoculation.

2) In a second example of an animal model, renal cells of primate or human origin which support JCV replication are transplanted into the flank or kidney subcapsular region of immunocompromised mice, e.g. COS-7 cells which are infected with JC virus inoculated into nude mice. Transplanted cells support in vivo replication of JCV at the site of inoculation.

3) In a third example, intravenous inoculation of huNSG or BLT mice containing functional circulating CD19⁺ human B cells with JC virus. Engrafted human B cells support replication of JCV in circulating blood and lymphoid tissues.

Mouse models with a humanized immune system such as immunocompromised and sub-lethally irradiated mice reconstituted with CD34⁺ human fetal liver cells (huNSG mice) or reconstituted with human thymus and human lymphocytes (BLT mice) produce functional circulating CD19⁺ human B cells. BLT mice inoculated with JCV remained asymptomatic following inoculation but JCV DNA was detected in both blood and urine and mice generated both humoral and cellular immune responses against JCV concomitant with expression of the immune exhaustion marker, PD-1, on lymphocytes consistent with a response to an infection. These mice are infected with JCV and used as a model for in vivo replication of the virus in B cells.

Other cells for transplantation include, without limitation those depicted in Table 1.

Table 1 shows cell transplantation models to support JCV replication and test therapeutics for PML

| | | | | viral | supports |
|---|---|---|---|---|---|
| | | species/ | route of | genes | JCV |
| cells/cell line | reference | cell type | injection | present | replication |
| Owl 26, 98, 586 | Major et al, 1984; | monkey, | i.c., s.c. | JCV | yes |
| | Major et al, 1987 | glial | | | |
| SVG/SVG-A | Major et al, 1985 | monkey, | i.c., s.c. | SV40 | yes |
| | Gee et al, 2003 | glial | | | |
| JCI/IMR-32 | Nukuzuma et al, 1995 | human, | i.c. | none | yes |
| | | neuroblastoma | | | |
| COS-7 | Gluzman et al, 1981 | monkey, | s.c. | SV40 | yes |
| | | kidney | | | |
| human glial | Kondo et al, 2014 | human, | i.c. | n/a | yes |
| progenitor | | glial | | | |
| cells | | | | | |
| human primary | n/a | human, | i.p. | n/a | yes |
| renal proximal | | kidney | | | |
| tubule endothelial | | | | | |
| cells | | | | | |
| humanized mice | Tan et al, 2013 | human, | i.v. | n/a | yes |
| (huNSG, BLT, etc.) | | lymphoid | | | |
| | | (B cells) | | | |
| HJC series | Raj et al, 1995 | hamster, | i.c., s.c. | JCV | no |
| | | glial | | | |
| BSB7 series | Krynska et al, 2000 | mouse, | i.c., s.c. | JCV | no |
| | | neuronal | | | |

### REFERENCES

Gee G V, Manley K, Atwood W J. Derivation of a JC virus-resistant human glial cell line: implications for the identification of host cell factors that determine viral tropism. Virology. 2003 Sep. 15; 314(1):101-9. PubMed PMID: 14517064.
Gluzman Y. SV40-transformed simian cells support the replication of early SV40 mutants. Cell. 1981 January; 23(1):175-82. PubMed PMID: 6260373.
Kondo Y, Windrem M S, Zou L, Chandler-Militello D, Schanz S J, Auvergne R M, Betstadt S J, Harrington A R, Johnson M, Kazarov A, Gorelik L, Goldman S A. Human glial chimeric mice reveal astrocytic dependence of JC virus infection. J Clin Invest. 2014 December; 124(12):5323-36. doi: 10.1172/JCI76629. PubMed PMID: 25401469; PubMed Central PMCID: PMC4348956.
Krynska B, Del Valle L, Gordon J, Otte J, Croul S, Khalili K. Identification of a novel p53 mutation in JCV-induced mouse medulloblastoma. Virology. 2000 Aug. 15; 274(1):65-74. PubMed PMID: 10936089.
Major E O, Miller A E, Mourrain P, Traub R G, de Widt E, Sever J. Establishment of a line of human fetal glial cells that supports JC virus multiplication. Proc Natl Acad Sci USA. 1985 February; 82(4):1257-61. PubMed PMID: 2983332; PubMed Central PMCID: PMC397234.
Major E O, Mourrain P, Cummins C. JC virus-induced owl monkey glioblastoma cells in culture: biological properties associated with the viral early gene product. Virology. 1984 Jul. 30; 136(2):359-67. PubMed PMID: 6087549.
Major E O, Vacante D A, Traub R G, London W T, Sever J L. Owl monkey astrocytoma cells in culture spontaneously produce infectious JC virus which demonstrates altered biological properties. J Virol. 1987 May; 61(5):1435-41. PubMed PMID: 3033272; PubMed Central PMCID: PMC254120.
Matoba T, Orba Y, Suzuki T, Makino Y, Shichinohe H, Kuroda S, Ochiya T, Itoh H, Tanaka S, Nagashima K, Sawa H. An siRNA against JC virus (JCV) agnoprotein inhibits JCV infection in JCV-producing cells inoculated in nude mice. Neuropathology. 2008 June; 28(3):286-94. doi: 10.1111/j.1440-1789.2007.00878.x. PubMed PMID: 18179406.
Nukuzuma S, Yogo Y, Guo J, Nukuzuma C, Itoh S, Shinohara T, Nagashima K. Establishment and characterization of a carrier cell culture producing high titres of polyoma JC virus. J Med Virol. 1995 December; 47(4):370-7. PubMed PMID: 8636705.
Raj G, Gordon J, Logan T, Hall D, Deluca A, Giordano A, Khalili K. Characterization of glioma-cells derived from human polyomavirus-induced brain-tumors in hamsters. Int J Oncol. 1995 October; 7(4):801-8. PubMed PMID: 21552907.
Tan C S, Broge T A Jr, Seung E, Vrbanac V, Viscidi R, Gordon J, Tager A M, Koralnik I J. Detection of JC virus-specific immune responses in a novel humanized mouse model. PLoS One. 2013 May 20; 8(5):e64313. doi: 10.1371/journal.pone.0064313. PubMed PMID: 23700470; PubMed Central PMCID: PMC3658964.

What is claimed:

1. A method of replicating human polyomaviruses in an animal comprising:
obtaining a cell permissive for human polyomavirus replication;
transplanting said cell into said animal;
inoculating said animal with the human polyomavirus; thereby,
replicating the human polyomavirus in the animal.

2. The method of claim 1, wherein the cell permissive for human polyomavirus comprises: human cells, humanized cells, primate cells, transformed cells, cell-lines, tumor cells, stem cells, hybrid cells, cells engineered to spontaneously shed a polyomavirus, or combinations thereof.

3. The method of claim 1, wherein the animal is a rodent.

4. The method of claim 3, wherein the rodent is immunocompromised.

5. The method of claim 1, wherein the replication of the polyomavirus is at the site of transplantation of the cell and/or is systemic.

6. A method of replicating a polyomavirus in a non-permissive animal comprising:
obtaining a cell permissive for human polyomavirus replication;
transplanting said cell into said animal;
inoculating said animal with the human polyomavirus; thereby,
replicating the human polyomavirus in the animal.

7. The method of claim 6, wherein the cell permissive for human polyomavirus comprise: human cells, humanized cells, primate cells, transformed cells, cell-lines, tumor cells, stem cells, hybrid cells, cells engineered to spontaneously shed a polyomavirus, or combinations thereof.

8. A method of identifying a candidate agent for inhibiting a human polyomavirus infection, or replication in vivo, comprising administering to an animal of claim 1 or 6, a candidate agent and assaying for human polyomavirus infection or replication.

9. A method of identifying candidate therapeutic agents for human polyomavirus and human polyomavirus-associated diseases comprising:
i.
a. administering a candidate therapeutic agent to an animal comprising transplanted cells permissive for human polyomavirus replication;
b. inoculating the animal with a human polyomavirus; and,
c. determining the presence or absence of any virus in the animal;
or
ii.
d. inoculating an animal comprising transplanted cells permissive for human polyomavirus replication;
e. administering to the animal a candidate therapeutic agent;
f. determining the presence or absence of any virus;
and,
selecting the candidate therapeutic agent which decreases viable human polyomavirus as compared to an inoculated control animal.

10. The method of claim 9, wherein the cell permissive for human polyomavirus comprises: human cells, humanized cells, primate cells, transformed cells, cell-lines, tumor cells, stem cells, hybrid cells, cells engineered to spontaneously shed a polyomavirus, or combinations thereof.

11. A non-human animal model comprising one or more transplanted cells or tissues permissive for replicating human polyomavirus and/or a human xenograft.

12. An animal model for identifying candidate therapeutic agents for the prevention and/or treatment of human polyomavirus and associated diseases thereof, comprising: one or more transplanted cells permissive for replicating human polyomavirus.
